(19) United States
(12) Patent Application Publication
(10) Pub. No.: US 2025/0127398 A1
(43) Pub. Date: Apr. 24, 2025

(54) SYSTEM AND APPARATUS FOR DETECTING A PSYCHOLOGICAL STATE OF A PERSON

(71) Applicant: FAWZAN ALQUDAIRI, SARASOTA, FL (US)

(72) Inventor: FAWZAN ALQUDAIRI, SARASOTA, FL (US)

(21) Appl. No.: 19/001,789

(22) Filed: Dec. 26, 2024

Related U.S. Application Data
(62) Division of application No. 17/305,089, filed on Jun. 30, 2021.

(51) Int. Cl. A61B 5/00; A61B 5/024; H04B 1/38

(52) U.S. Cl. CPC A61B 5/0004; A61B 5/02438; A61B 5/681; A61B 5/7221; H04B 1/38

(57) ABSTRACT

A system and method for detecting the psychological or physical state of a person. The system includes a signal transmitter coupled with a signal receiver, a signal receiver coupled to a signal transmitter, and a sensor element coupled to a body capable of determining the heart rate of the wearer. A first transmitter transmits a request for the physiological state of the wearer of a sensor to a signal receiver. The signal receiver, preferably coupled to the sensor, obtains the heart rate from the sensor, which measures and transmits back to the receiver a heart rate, which in turn determines the physiological state of the wearer of the sensor to determine the degree of acceptance, satisfaction, rejection or anger.

### CROSS-REFERENCE TO RELATED APPLICATIONS

This application is a divisional of a U.S. patent application Ser. No. 17/305,089 filed on Jun. 30, 2021, which is incorporated herein by reference in its entirety.

### FIELD OF INVENTION

The present invention relates to a system and method for the detection of the psychological or physical state of an individual, and more specifically to a system comprising a first transmitter coupled to a receiver, a receiver coupled to a transmitter, and a sensor disposed on the body of a wearer.

### BACKGROUND

The present teachings in some embodiments are predicated upon systems and methods for detecting the psychological or physical state of a signal receiver wearer. More specifically, the current invention relates to the field of transmitting and receiving signals, wherein the received signal contains a text message, an audio or video clip, a picture, or similar stimulant, whether it is between two people engaging directly in a mutual conversation or through a communication platform. The invention is applicable wherever a receiver and a transmitter are this signal.

In the present, signal transmitters are not adapted to receive the correct and accurate expression by a receiver of a signal stimulant. Typically, the recipient's expression is often manual and does not express the opinion of the signal receiver accurately. It is thus the objective of this invention to improve this signal process by the use of the system and apparatus that will be herewith disclosed in the descriptions of the current invention. The current invention would improve the attractiveness and flexibility of signal receivers to be able to accurately receive the response of a receiver according to the degree of comfort and conviction of the material displayed in the transmitted signal.

It is thus an object of the current invention to make it possible for any such party participating in the system anticipated by the current invention to know the opinion or reaction of the receiver to this signal in a real and realistic way without forgery or courtesy. It may also be desirable for a receiver of a communication to know the true nature of their feelings and their opinion, without external influences that sometimes compel them to express an insincere opinion. The current invention specifies the degree of satisfaction, acceptance, or rejection, and expresses the opinion realistically, to aid in making decisions that will be taken based on the true state of the receiver of the signal.

In prior art implementations, the expressed opinion for a received signal may be merely courtesy and the opinion may change easily because it was not a real opinion from the beginning, and this may affect decisions that will be taken based on the responses to these signals. However, in the current invention the expressed liking or dislike towards a signal received will be accurate and may further be adapted as a percentage dependent upon the measured heartbeat at the time of receiving the signal. Further, the percentage may change depending on the measured heartbeat and can be deduced as a true reflection of what the receiver feels towards the received signal, and as a consequence help in making the right decision.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The following summary is an explanation of some of the general inventive steps for the system, method, architecture, and apparatus in the description. This summary is not an extensive overview of the invention and does not intend to limit the scope beyond what is described and claimed as a summary.

In summary, the present invention, in some embodiments thereof, relates to the detection of the psychological or physical state of a signal receiver wearer, wherein it is provided a computer-implemented system and methods for the detection of the psychological or physical state of a signal receiver wearer, and specifically to a system comprising a first transmitter coupled to a receiver, a receiver coupled to a transmitter and a sensor disposed on the body of a wearer. In an exemplary embodiment, the current invention uses the heartbeat of the receiver, wherein a sensor is placed on the body of the wearer, and the output is processed to determine the level of the heartbeat, and accordingly know their feeling of rejection, anger, or satisfaction and approval of the received signal and the degree of approval by a percentage, preferably taking into account any changes owing to physical activity at the time of receiving the signal or in such cases where chronic diseases and medical history are involved. Preferably, chronic illnesses and physical activity information may further be perceived and processed by a sensor fixed around the wrist of the signal receiver.

### BRIEF DESCRIPTION OF DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a diagram illustrating the architecture of the system, according to the description of the current invention.

FIG. 2 is a flowchart illustrating the operation of a signal-transmitting device, according to the description of the current invention.

FIG. 3 is a flowchart illustrating the operation of a signal-receiving device, according to the description of the current invention.

FIG. 4 is a flowchart illustrating the operation of a heart rate sensing device disposed on a receiver's body, according to the description of the current invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Hereinafter, the preferred embodiment of the present invention will be described in detail with reference to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms to describe the invention in the best way.

It is to be understood that the form of the invention shown and described herein is to be taken as a preferred embodiment of the present invention, so it does not express fully the technical spirit and scope of this invention. Accordingly, it should be understood that various changes and modifications may be made to the invention without departing from the spirit and scope thereof.

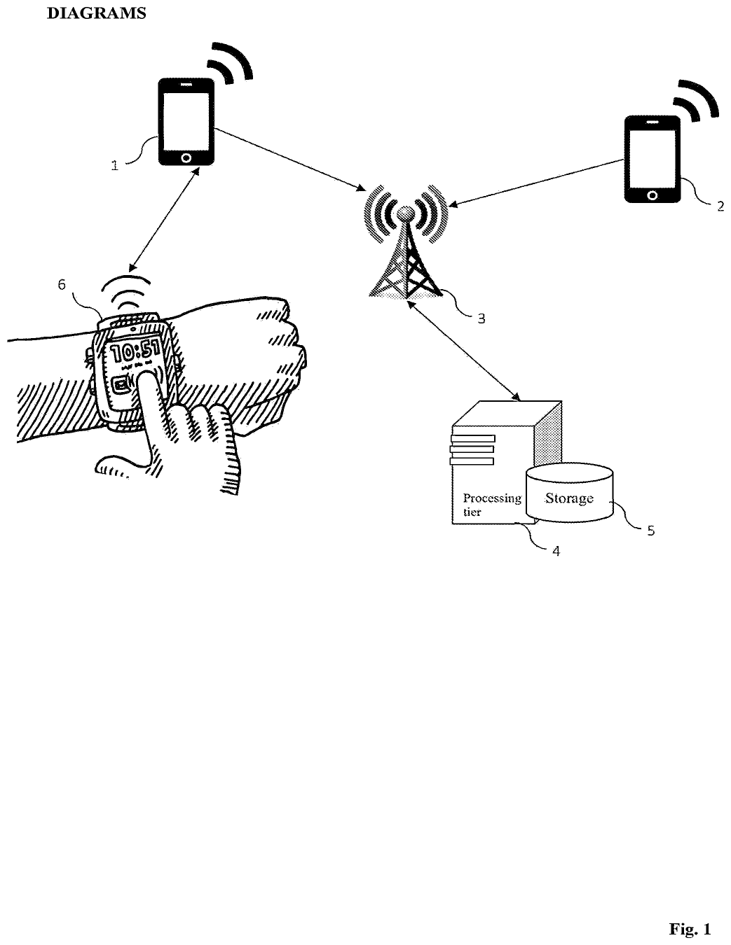

The first exemplary embodiment according to FIG. 1 of the diagrams is an exemplary illustration of a system of the description of the current invention. In the diagram illustrated as 2, is a signal transmitting device, preferably comprising at least in part of a signal generation mechanism, a signal transmitter, a signal receiver, and a means to display a received signal. In some embodiments, the device 2 may embody a portable computing device. Preferably, the signal may be incorporated in a text message, an audio or video clip, a picture or similar stimulant, electromagnetic wave, mechanical or any such, which may be transmissible between any number of devices within a local area network such as with Bluetooth, Wi-Fi, NFC or a wide area network such as with the internet, mobile broadband, directly between devices or through an intermediary processing platform comprising at least in part of a processing tier 4 and a data storage 5.

Illustrated in the same FIG. 1, is a network 3, which may embody the internet, Wi-Fi, Bluetooth, local area network, wide area network, fiber optic, mobile broadband, 4G, 5G, 6G, or any such implements that would enable the transmission of packets of data between any number of devices, and specifically in the context of the current invention, the transmission of signals between the devices participating in the implementation of the invention. The signal transmitting device 2, may suitably comprise of at least a memory, one or more processors, a network interface, and a display apparatus and would be configured with a suitable computer program product comprising of a sequence of instructions on its memory, which when executed by said processor or processors, would make the device to transmit a signal to a receiver, receive the status of the receive at the time of transmission and display the psychological state of the receiver at the transmission and in reaction to the signal.

Further still in the same FIG. 1 is a device 1, which according to the current invention embodies a signal-receiving device, preferably comprising at least in part of signal reception mechanism, a signal receiver, and a signal transmitter. Preferably, the signal may comprise of a text message, an audio or video clip, a picture or similar stimulant, electromagnetic wave, mechanical, or any such, which may be transmissible between any number of devices within a local area network such as with Bluetooth®, Wi-Fi, NFC or a wide area network such as with the internet, mobile broadband, directly between devices or through an intermediary processing platform comprising at least in part of a processing tier 4 and a data storage 5.

The signal-receiving device 1, may suitably comprise of at least a memory, one or more processors, a network interface, and a display apparatus and would be configured with a suitable computer program product comprising of a sequence of instructions on its memory, which when executed by said processor or processors, would make the device to receive a signal from a transmitter, transmit the status of the receiver at the time of receiving the signal and transmit the psychological state of the receiver at the reception of and in reaction to the signal. Further still in the same FIG. 1, is a sensor 6, which according to the current invention may embody a smart watch capable of measuring the heart rate of the wearer. Preferably, the signal receiver's level of heartbeat in normal conditions is saved in the sensor or a receiver device 1 coupled to said sensor, and as such, it can be established through a suitable device configuration, such as using a computer program product comprising of a sequence of instructions configured on the device memory, when the state of anger or dissatisfaction the level of the heartbeat rises and thus expresses nonsatisfaction with the received signal. The improved technical utility that characterizes the disclosed approach would make its possible application to a large segment of users, so it can be said that one general objective of this invention is to find a method for determining the opinion about the received signal more accurately, automatically, new and its industrial application provides a better utility than the prior art manual methods.

The disclosed system and methodology are an obvious improvement to the currently recognized manual methods, which include a configuration for either the like or dislike of a received signal and which cannot be guaranteed as accurate because the reporting may be wrong or not express the true opinion of the receiver. It is noteworthy that prior art descriptions do not provide for any accurate measurement of liking or disliking, which provides the user with limited options to express an opinion that sometimes lacks credibility or transparency.

In some embodiments, a processing tier 4 comprising at least in part of a memory, a processor, and a storage device 5, may be provided as an intermediary medium for the transmission of signals between devices 1 and 2. It may further be adapted for the accessible storage of a signal, which forms part of the object of transmission including but not limited to a text message, an audio or video clip, a picture or similar stimulant, an electromagnetic wave, mechanical or any such, which may be transmissible between any number of devices within a network of any type.

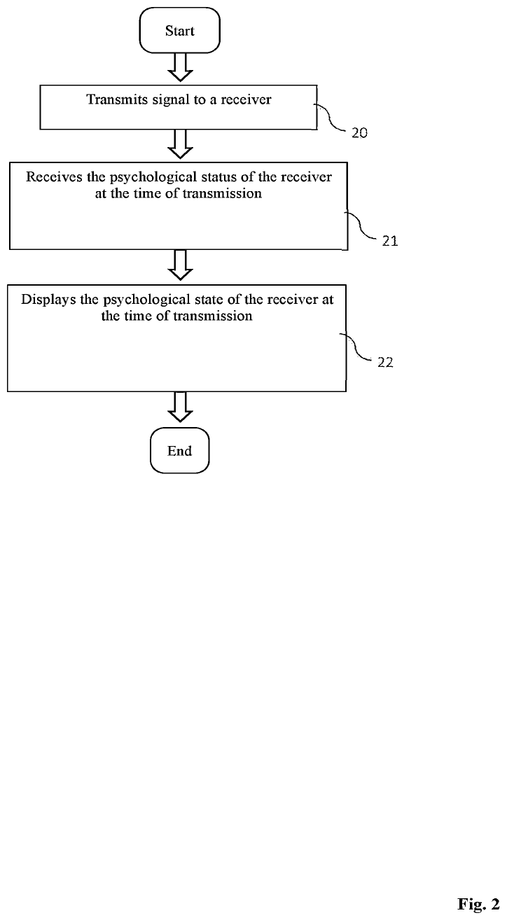

In an exemplary embodiment as provided in FIG. 2 of the diagrams is an exemplary illustration of the operation of a signal transmitting device. A suitably configured signal transmitting device transmits a signal to a receiver device in a network, local, or wide area as in step 20. In step 21, the signal-transmitting device may receive the psychological state of the receiver at the time of transmission of the signal. This psychological state of the receiver may be received after the determination of the level of the heartbeat of the receiver by a corresponding recipient device, and accordingly know their feeling of rejection, anger, or satisfaction and approval of the received signal and the degree of approval by a percentage, preferably taking into account any changes owing to a physical activity at the time of receiving the signal or in such cases where chronic diseases and medical history are involved. In step 22, the method entails a further display of the psychological state of the receiver on a suitable display apparatus of the signal-transmitting device.

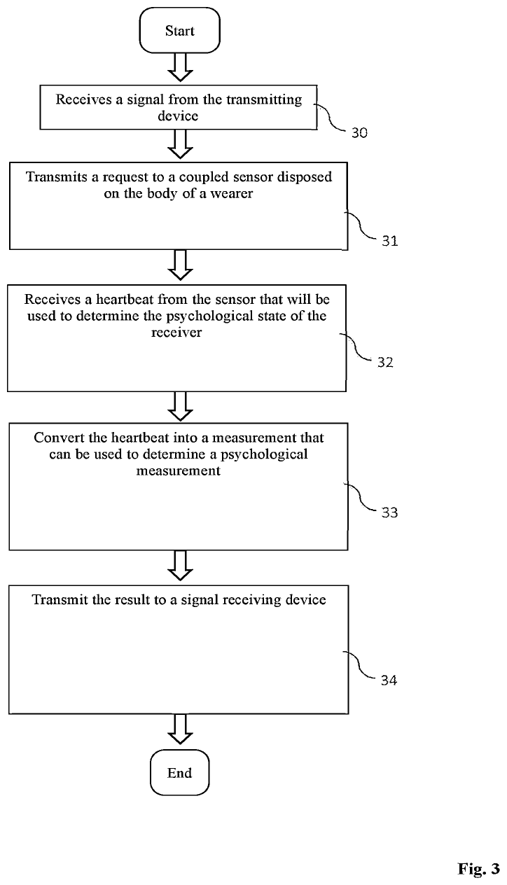

According to an embodiment according to FIG. 3 of the diagrams is an exemplary illustration of the operation of a signal-receiving device. A suitably configured signal receiving device such as 1, in FIG. 1, receives a signal from a suitably configured signal transmitting device such as 2, in FIG. 1 in step 30. The signal-receiving device subsequently in step 31, transmits a request to a coupled sensor disposed on the body of a receiver for a measured heart rate. Next, in step 32, the signal-receiving device receives the heart rate from the coupled sensor, as illustrated in the FIG. 1 above, wherein the received heart rate may be used to determine the psychological state of the receiver. Subsequently, in the step 33, the received heart rate or heartbeat measurement is converted into a psychological state of the receiver of rejection, anger, or satisfaction and approval of the received signal and the degree of approval by a percentage, preferably taking into account any changes owing to a physical activity at the time of receiving the signal, or in any such cases where chronic diseases and medical history are involved. Finally, in the step 34, there is a transmission of the determined psychological state to a signal-transmitting device. Preferably, the transmission is performed via any such network implementation.

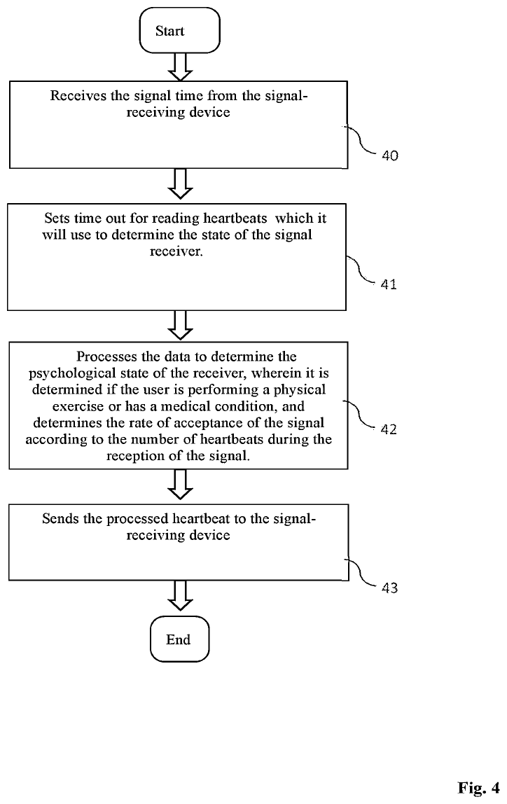

In a further and final exemplary embodiment according to FIG. 4 of the diagrams is an illustration of the operation of a heart rate sensing device or sensor disposed on a receiver's body. Beginning at the step 40 of the operation is receiving a request at the time of a signal transmission for the measured heart rate. Subsequently, in the step 41, the sensor or heart rate sensing device disposed on or inside a receiver's body performs an action of reading the heart rate that will be used to determine the receiver's psychological state, the device may further set a time-out for the reading/measurement of the receiver's heart rate.

Subsequently, in the step 42, the device processes the heart-rate measurement data to determine the psychological state of the receiver, wherein it is determined if the user is performing a physical exercise or has a medical condition and determines the rate of acceptance of the signal according to the number of heartbeats during the reception of the signal. Finally, in 43, the processed heartbeat measurement is transmitted to the signal-receiving device.

In order to improve the prior art implementations, the current invention provides a distinctive method for signal-receiving and transmitting devices to be used more effectively through this signal-receiving sensor, which is disposed in receiving devices. Further, the method disclosed for the current invention's receiving sensor is new, inexpensive, and generally very accessible to the public to provide the best solution for determining the accuracy of responses at the signal receiver.

According to an exemplary embodiment, the signal-transmitting device comprises a signal receiver, capable of receiving the psychological state measured and determined at the receiver.

According to an exemplary embodiment, the signal receiver device comprises a signal transmitter, capable of transmitting the psychological state measured.

According to an exemplary embodiment, the heart-rate sensing device or sensor may be configured capable of measuring the heart rate, while taking into account any variations within a specified period, such as 24 hours, from which the minimum number of heart beats per minute and the maximum number of heart beats per minute may be determined, and which would help deduce the normal heartbeats level of the signal receiver and as such enable the exclusion of variations caused by physical exercise at the time of receiving a signal, and further exclude variations caused by a medical condition.

According to an exemplary embodiment, the heart-rate sensor will comprise of a measurement history of the signal receiver to determine variations caused by physical activity, such as sport, at the time of receiving the signal, and as such aid in the processing of measured data to determine the correct measurement of the heartbeat and transmits it to the signal-receiving device.

According to an exemplary embodiment, the receiver of the heart-rate measurements may be adapted to process the heartbeat or heart-rate measurement in such a manner that the correct reading is determinable when receiving the signal, by factoring interruptions during measurement and historical measurements, wherein the correct psychological or state of the signal receiver may be determined more accurately.

The foregoing is provided for the purposes of illustrating, explaining, and describing embodiments of the present invention. Modifications and adaptations to these embodiments will be apparent to those skilled in the art and may be made without departing from the scope or spirit of the invention.

### INDUSTRIAL APPLICATION

The current invention technology is applicable to signal receivers, and more specifically, the method of applying to industry relates to the detection of the psychological state of a receiver of a signal to improve the overall accuracy of systems that detect the psychological state of a receiver of a signal.

What is claimed is:

1. A method for detecting a psychological state of a user, the method comprising:
transmitting a signal by a transmitting unit of a first device to a receiving unit of a second device;
collecting psychological state data by a wearable device, wherein the wearable device comprises:
a sensor configured to detect a psychological state of the user, and
a transmitting unit configured to transmit the psychological state data to the second device;
receiving the psychological state data by the receiving unit of the second device from the transmitting unit of the wearable device;
transmitting the psychological state data by the transmitting unit of the second device to a receiving unit of the first device; and
displaying the psychological state on the first device, wherein the displayed psychological state corresponds to the psychological state of the user at a time of signal transmission from the first device to the second device.

2. The method of claim 1, wherein the sensor is configured to detect heartbeat rate, and the psychological state is determined from the heartbeat rate.

3. The method of claim 2, wherein the method further comprises:
detecting, by the sensor, the heartbeat rate of the user; and
determining a physical activity of the user at the time of detecting the heartbeat rate by the sensor;
determining a medical condition of the user at the time of detecting the heartbeat rate by the sensor; and
determining the psychological state by processing the measured heartbeat rate, the physical activity, and the medical condition.

4. The method of claim 1, wherein the signal comprises of a text message.

5. The method of claim 1, wherein the signal comprises of an audio file.

6. The method of claim 1, wherein the signal comprises of a video file.

7. The method of claim 1, wherein the signal comprises of a picture.

8. The method of claim 1, wherein the signal is in a form of an electromagnetic wave.
